(19) United States
(12) Patent Application Publication
(10) Pub. No.: US 2008/0032410 A1
(43) Pub. Date: Feb. 7, 2008

(54) Labeling for identification of proteins and other macromolecules

(75) Inventor: Richard Moerschell, Concord, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Bio-Rad Laboratories, Inc., Hercules, CA (US)

(21) Appl. No.: 11/879,280

(22) Filed: Jul. 16, 2007

Related U.S. Application Data
(60) Provisional application No. 60/832,169, filed on Jul. 19, 2006.

(51) Int. Cl. G01N 33/68

(52) U.S. Cl. 436/86

(57) ABSTRACT

A method for identifying one or more proteins or other macromolecules comprises contacting the proteins or other macromolecules or a material that contains them with soluble fluorescent labels and/or Raman labels that bind to at least two different functional groups, side chains or moieties that may be present on or in proteins or other macromolecules, determining the ratio of the two or more functional groups, side chains or moieties, and comparing that ratio with ratios of the said functional groups, side chains or moieties in known proteins or other macromolecules. Kits for carrying out such a process contain fluorescent and/or Raman labels that bind to two or more of said functional groups, side chains or moieties, a database or other medium containing information respecting the ratio of said functional groups, side chains or other moieties in known proteins or other macromolecules, and software for comparing the measured ratio of labels to the predicted ratio of labels to identify the proteins or other macromolecules.

### BACKGROUND OF THE INVENTION

This invention relates to methods for identifying proteins and other macromolecules that involve the use of techniques such as gel electrophoresis, and chromatography, and more specifically to methods and kits for labeling proteins and other such molecules for more optimal identification, particularly proteins found in a spot on a gel formed through the use of electrophoresis.

A number of techniques are available for use with gel electrophoreses (e.g., SDS-PAGE) in identifying protein side chains and other features. For example, some stains can be used to differentiate or track modified proteins such as phosphorylated proteins and glycosylated proteins, as well as unmodified proteins. Such stains include the Pro-Q® Diamond and Emerald and Sypro® Ruby fluorescent gel stains available from Invitrogen. Such stains are not covalently bound to the proteins and are only used after electrophoresis.

Proteins can be covalently labeled prior to electrophoresis. Targeting of active sites of enzymes with irreversible inhibitors such as ABPs (activity based probes) available from ActivX Biosciences, Inc., a subsidiary of Kyorin Pharmaceutical Co. Ltd (Japan)] Briefings in Functional Genomics and Proteomics, Vol. 1, pp. 151-158 (2002)] has been used to label and identify different types of enzymes on gels. Alternatively, unstained and unlabeled proteins can be visualized in gels using the LINF (laser induced native fluorescence) technique which detects proteins having tryptophan residues, for example with technology supplied by Lavision BioTec (International patent application WO2004/013625). Metabolic labeling with C¹⁴ tyrosine and S³⁵ methionine has been used to identify the ratio of these amino acids in protein spots on 2D gels [Garrels et al., Electrophoresis 18:1347 (1997)].

The techniques mentioned above are used to label a protein or another type of macromolecule via attachment to a particular functional group so that the protein may be tracked through various chemical or biological processing steps. Typically only a single label is needed to track or differentiate the protein in question from others in the system. Gel stains such as those mentioned above are also used for identification of unknown proteins. This is accomplished by comparing a stained get spot with a known spot on a control gel. However, such comparison is done visually, and thus has built-in inaccuracies.

Mass spectrometric (MS) analysis is also used to identify unknown proteins, at times followed by visual comparison of spots. However, this technique, while providing greater accuracy than comparison of gel spots alone, requires expensive equipment and still includes factors of visual identification of gel spots that can produce inaccuracies or uncertainties in identifications. Garrels et al., supra, using radioactive isotope-labeled tyrosine and methionine, together with information on the molecular weight and isoelectric point (pI) of proteins, codon bias and a yeast protein database, was able to identify at least 80% of spots on a gel that represented abundant proteins. However, in many cases identification of proteins is carried out using samples obtained from human patients. A sample of, for instance, a biological body fluid, is obtained from a patient having or suspected of having a certain condition. That sample is then run on an electrophoresis gel and compared with a sample from a healthy individual, to ascertain which proteins are elevated and/or depressed in the patient as compared with one who is healthy. Radioactive isotope labeling cannot be carried out in such situations since the isotopes cannot be fed to humans.

It would be desirable to have relatively quick, relatively inexpensive and relatively accurate methods for identifying unknown proteins and other macromolecules.

### BRIEF SUMMARY OF THE INVENTION

In brief, this invention comprises a method for identifying one or more proteins or other macromolecules that comprises contacting the proteins or other macromolecules, or a material that contains them, with soluble fluorescent labels and/or Raman labels that bind to at least two different functional groups, side chains or moieties that may be present on or in proteins, determining the ratio of the two or more functional groups, side chains or moieties, and comparing that ratio with ratios of the said functional groups, side chains or moieties in known proteins or macromolecules. The invention also includes a kit for carrying out such a method, containing fluorescent labels and/or Raman labels that bind to two or more of said functional groups, side chains or moieties, a database or other medium containing information respecting the ratio of said functional groups, side chains or moieties in known proteins or other macromolecules, and software for comparing the measured ratio of labels to the predicted ratio of labels to identify the proteins or other macromolecules.

### DETAILED DESCRIPTION OF THE INVENTION

In brief, this invention comprises a method for identifying one or more proteins or other macromolecules that comprises contacting the proteins or other macromolecules, or a material that contains them, with soluble fluorescent labels and/or Raman labels that bind to at least two different functional groups, side chains or moieties that may be present on or in proteins, determining the ratio of the two or more functional groups, side chains or moieties, and comparing that ratio with ratios of the said functional groups, side chains or moieties in known proteins or macromolecules. The invention also includes a kit for carrying out such a method, containing fluorescent labels or Raman labels that bind to two or more of said functional groups, side chains or moieties, a database or other medium containing information respecting the ratio of said functional groups, side chains or moieties in known proteins or other macromolecules, and software for comparing the measured ratio of labels to the predicted ratio of labels to identify the proteins or other macromolecules.

In comparison to previously used techniques, the procedure of this invention can provide results less expensively and/or quicker and/or more reliably, depending on which technique the comparison is made with. As compared to MS alone, the procedure of the invention is less expensive and nearly as accurate. As compared with visual comparison of gel spots, the inventive procedure is more reliable, quicker and/or less expensive as well.

The functional groups, side chains or moieties in proteins that may be labeled with fluorescent labels according to this invention include alpha and/or epsilon amino groups, carboxyl groups, cysteine, tryptophan, and other amino acids, as well as post-translational and co-translational modifications.

While the description of this invention is set out in terms of labeling and identifying proteins, the same techniques may be used for identifying other types of macromolecules such as sugars, lipids, and nucleic acids. The types of functional groups or other moieties for which labels will be provided will depend on the nature of the macromolecule and the types of groups such molecules typically may contain. However, the provision of a kit that contains at least two, and preferably at least three, fluorescent or other types of labels as described herein for different groups, an appropriate database or other medium of information and appropriate software for performing the identification, and the identification of the macromolecule by ascertaining ratios of the labeled groups, applies to molecules other than proteins as well. For example, reducing sugars on carbohydrates can be derivatized as well by periodate oxidation. Lipids can be labeled with aldehyde-tagged molecules [McMillen et al., “Identifying regions of membrane proteins in contact with phospholipid head groups: covalent attachment of a new class of aldehyde lipid labels to cytochrome c oxidase.” Biochemistry 1986; 25(1):182-93)].

While the invention is described herein in terms of the use of fluorescent labels and identification of proteins using that fluorescence, other labels may be used, particularly Raman labels (which can be detected using, for example, Raman spectrometers). A given method or kit may comprise fluorescent labels, Raman labels, or a combination of both. If both types of labels are included in a kit, or in a method according to the invention, the labels should be selected so that the emissions from one do not interfere with emissions from the other.

If the fluorescent labels used in the invention are relatively water-soluble, more labels can be bound to a macromolecule without appreciably reducing the overall solubility of the conjugate. Any known fluorescent or Raman label for the functional groups, side chains or moieties can be used in the procedures of the invention, provided that it either does not significantly affect the molecular weight or isoelectric point of proteins or macromolecules in general or affects them in a consistent and predictable fashion. For example, CyDyes®, available from GE Healthcare, may be used, as well as labels available from Invitrogen (formerly sold by Molecular Probes Co.) or as described in U.S. published patent application 20040248203 of Dratz et al. Suitable labels for amino and thiol (such as found on cysteine groups) are found in U.S. published patent application 20040248203. Fluorescent labels for carboxyl groups are commercially available. Alternatively, labels for carboxyl groups could be prepared by replacing an amino or thiol binding ligand on a label with a carboxyl binding ligand such as a carbodiimide. Suitable labels for tryptophan include TCE, available from SigmaAldrich Co. (Ladner C L, Yang J, Turner R J, Edwards R A., “Visible fluorescent detection of proteins in polyacrylamide gels without staining.” Anal Biochem. 2004 Mar. 1; 326(1): 13-20.).

Other known means of identifying proteins or other macromolecules can be used together with the methods of this invention that employ fluorescent and/or Raman labels. For example, phenylalanine, tryptophan and tyrosine have natural or native fluorescence, and thus the use of the above-described LINF or other technique for detecting such fluorescence can be used together with labels of this invention to assist in identification of a protein. Similarly, Raman spectroscopy may be used together with the labels and optionally together with identification of natively fluorescent amino acids in identifying a given protein.

A kit according to the invention will contain appropriate amounts of two or more fluorescent and/or Raman labels according to the invention for groups mentioned above, preferably three or more such labels. Currently no apparatus is available that could be used to detect both of these types in the same step, so if a combination of fluorescent and Raman labels is used in the invention they should be selected so as not to interfere with each other's detection. However, the apparatus or equipment used to identify the proteins or other macromolecules is not part of this invention, which preferably uses standard apparatus.

The labels may be contained in separate vials or other containers, or may be provided as a mixture of labels in a single vial. Also optionally included in the kit may be a database on CD-ROM or other suitable device or medium that contains data on ratios in proteins of the groups for which labels are included. The kits will also typically contain other items that are appropriate for kits of these types, such as EDAC and other reagents that may be needed for the labeling reaction. By contacting the proteins or other macromolecules, or the sample that contains them with the labels, then submitting the sample to gel electrophoresis, chromatography, or other suitable separation or identification procedure and comparing the ratio of the stained groups to information in the database or other medium using a computer, a reasonably accurate identification of a protein or proteins or other macromolecules in the sample can be obtained, or the identity of the protein or other macromolecule could at least be narrowed to a smaller number of possibilities. If desired, MS can be used to confirm or further narrow the likely identification of the protein(s) or other macromolecule(s).

In determining which proteins are elevated or depressed in a patient being diagnosed for a disease or condition, a sample of a bodily biological fluid (e.g., blood, urine, saliva, etc.) is obtained from the patient and compared with a similar sample from a healthy individual. Both samples are treated with labels contained in the kits and the resulting labeled proteins are identified and compared, thus providing an indication of elevation or depression of a particular protein or proteins. The use of MS is not necessary in a procedure of this type but could be used if desired. Of course, as discussed above, the procedures and kits of the invention may be used to identify proteins per se, in samples of liquids, which may include biological fluids or other liquids.

It is known that proteins stoichiometrically labeled with the fluorescent labels described herein can be less soluble than the corresponding unlabeled proteins. In addition, labeling of functional groups located close to one another on a protein molecule can impair or partially mask detection of the labels. Accordingly, in a preferred embodiment, the labeling is deliberately carried out in less than a stoichiometric manner. As it is not known in advance what groups are ultimately present to be labeled, or how many groups are present in a protein, this can be effectively done by essentially diluting the amount of a given fluorescent or other label, by combining it with a larger amount of another substance that binds to the same functional group, side chain or moiety, but that does not serve a labeling function, e.g., is not readily detectable by available instrumentation. The second substance may be, for example a compound that provides a blocking or protecting group for a functional group. For example, a compound that furnishes an acetyl (Ac), trifluoroacetyl, benzoxycarbonyl (Cbz), ter.-butoxycarbonyl (Boc), allyloxycarbonyl (Aoc), 9-fluorenylmethyloxycarbonyl (Fmoc), or phthaloyl group may be used to bind to amino groups of proteins in place of a fluorescent label. Similarly, a compound that acylates or alkylates a carboxyl group, such as by formation of an ester or ether using, e.g., acetyl, benzoyl, trityl, or substituted silyl groups, may be used to bind to carboxyl groups. Alternatively, for each fluorescent label, a non-fluorescent counterpart could be made based on the same derivitization chemistry where the fluor is replaced with a non-fluorescent moiety which has similar effect on mobility of the macromolecule but has an equal or better solubility than the fluorescent label.

When a fluorescent label diluted with a non-fluorescent label, or a Raman label similarly diluted is employed in a kit or procedure of the invention, the fluorescent or Raman label may be present in as little as 5 mole % of the total content of groups that label or bind to the functional group, side chain or moiety in question. Nonetheless, such an amount is still sufficient to provide labeling that can serve to identify the protein in question with reasonable accuracy, via determining a ratio of labeled groups. In preparing a kit that will contain one or more “diluted” fluorescent or Raman labels, the extent to which a label can be diluted and still be effective in the procedures of this invention can be tested by preparing a lysate of an appropriate known protein and running it on a gel to see whether it can be accurately identified.

From the foregoing description, various modifications and changes in the compositions and methods of this invention will occur to those skilled in the art. All such modifications coming within the scope of the appended claims are intended to be included therein.

All publications, including but not limited to patents and patent applications, cited in this specification are herein incorporated by reference as if each individual publication were specifically and individually indicated to be incorporated by reference herein as though fully set forth.

What is claimed is:

1. A method for identifying one or more proteins or other macromolecules that comprises contacting the proteins or other macromolecules, or a material that contains them, with two or more soluble fluorescent labels and/or Raman labels that bind to at least two different functional groups, side chains or moieties that may be present on or in proteins or other macromolecules, determining the ratio of the two or more functional groups, side chains or moieties, and comparing that ratio with ratios of the said functional groups, side chains or moieties in known proteins or other macromolecules.

2. A method according to claim 1 in which the labels comprise fluorescent labels.

3. A method according to claim 1 in which the labels comprise Raman labels.

4. A method according to claim 1 in which the one or more proteins or other macromolecules are contacted with labels that bind to at least three different functional groups, side chains or moieties.

5. A method according to claim 1 further comprising determining native fluorescence of amino acids that may be comprised in the one or more proteins or other macromolecules.

6. A kit for identifying one or more proteins or other macromolecules comprising two or more fluorescent labels and/or Raman labels that bind to at least two different functional groups, side chains or moieties that may be present on or in proteins or other macromolecules, a database or other medium containing information respecting the ratio of said functional groups, side chains or moieties in known proteins or other macromolecules, and software for comparing the measured ratio of labels to the predicted ratio of labels to identify the proteins or other macromolecules.

7. A kit according to claim 6 in which the labels comprise fluorescent labels.

8. A kit according to claim 6 in which the labels comprise Raman labels.

9. A kit according to claim 6 in which the labels bind to at least three different functional groups, side chains or moieties.

10. A kit according to claim 6 in which the labels are diluted with other substances that bind to the same functional groups, side chains or moieties but that do not serve a labeling function.
